(19) United States
(12) Patent Application Publication
(10) Pub. No.: US 2026/0060745 A1
(43) Pub. Date: Mar. 5, 2026

(54) RF GUIDEWIRE NEEDLE WITH PASSIVE ELECTRODE SHEATHING DESIGN

(71) Applicant: Boston Scientific Medical Device Limited, Galway (IE)

(72) Inventor: Matthew DiCicco, Toronto (CA)

(21) Appl. No.: 19/313,723

(22) Filed: Aug. 28, 2025

Related U.S. Application Data
(60) Provisional application No. 63/688,740, filed on Aug. 29, 2024.

(51) Int. Cl. A61B 18/14; A61B 18/00; A61B 18/12; A61B 18/16; A61B 90/00

(52) U.S. Cl. CPC A61B 18/1492; A61B 18/1206; A61B 90/03; A61B 90/39; A61B 2018/00077; A61B 2018/00083; A61B 2018/00178; A61B 2018/00184; A61B 2018/00351; A61B 2018/00577; A61B 2018/162; A61B 2090/034; A61B 2090/3966

(57) ABSTRACT

A perforation device having a passive electrode sheathing design includes an elongate body having a proximal portion including a proximal end and a distal portion including a distal end. At least one electrode is located at the distal end. The at least one electrode is configured to receive energy for piercing a target tissue. At least one conductor extends from the proximal end to the at least one electrode. The at least one conductor is configured to electrically connect the at least one electrode to a control system. A movable insulation sheath is configured to move from a first position to a second position, wherein the first position covers the at least one electrode and the second position exposes the at least one electrode.

### CROSS REFERENCE

This application claims priority to U.S. Provisional Application No. 63/688,740 entitled “RF GUIDEWIRE NEEDLE WITH PASSIVE ELECTRODE SHEATHING,” filed Aug. 29, 2024, which is herein incorporated by reference in its entirety.

### TECHNICAL FIELD

The present disclosure relates to medical systems and methods for ablating tissue in a patient. More specifically, the present disclosure relates to medical systems and methods for protecting adjacent tissue from damage during an ablation procedure.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Additionally, RF ablation can be used to help introduce devices through tissue. For example, RF energy can be delivered to a tip electrode of a perforation device in order to aid in the cutting of tissue. Use of RF energy allows the perforation of tissue without the need for a substantial application of force. In some instances, uncontrolled delivery of energy to a tip electrode can lead to damage to tissue adjacent a target area.

### SUMMARY

Example 1 is a perforation device having a passive electrode sheathing design. The device includes an elongate body having a proximal portion including a proximal end and a distal portion including a distal end. At least one electrode is located at the distal end. The at least one electrode is configured to receive energy for piercing a target tissue. At least one conductor extends from the proximal end to the at least one electrode. The at least one conductor is configured to electrically connect the at least one electrode to a control system. A movable insulation sheath is configured to move from a first position to a second position, wherein the first position covers the at least one electrode and the second position exposes the at least one electrode.

Example 2 is the device of Example 1, further comprising a platinum band located proximal of the at least one electrode.

Example 3 is the device of Example 1 or 2, further comprising a spacer located proximal of the at least one electrode.

Example 4 is the device of Example 3, further comprising a radiopaque coil, wherein a proximal face of the spacer is configured to abut a distal end of the radiopaque coil.

Example 5 is the device of Example 3, wherein the spacer is cylindrical and formed of a non-conductive material.

Example 6 is the device of Example 5, wherein the spacer is a formed of a ceramic material.

Example 7 is the device of Example 1, wherein the movable insulation sheath includes a compressible portion.

Example 8 is the device of Example 3, wherein the spacer is affixed to the movable insulation sheath.

Example 9 is the device of Example 8, further comprising a stop located on the at least one conductor, the stop being configured to abut the spacer to prevent further proximal movement of the moveable insulation sheath.

Example 10 is the device of any of Examples 1-9, further comprising a radiopaque coil located in the distal portion.

Example 11 is the device of Example 10, wherein the movable insulation sheath is attached to a proximal end of the radiopaque coil, a distal end of the radiopaque coil, or both the proximal end and the distal end of the radiopaque coil.

Example 12 is the device of any of Examples 1-11, wherein the distal portion includes a pre-formed curve.

Example 13 is the device of Example 12, wherein the pre-formed curve includes a J-shape or a spiral shape.

Example 14 is the device of any of Examples 1-13, wherein the distal portion includes first diameter, and the distal end includes a second diameter smaller than the first diameter.

Example 15 is the device of any of Examples 1-14, wherein the movable insulation sheath includes a distal face configured to contact a target tissue.

Example 16 is a perforation device having a passive electrode sheathing design. The device includes an elongate body having a proximal portion including a proximal end and a distal portion including a distal end. At least one electrode is located at the distal end. The at least one electrode is configured to receive energy for piercing a target tissue. At least one conductor extends from the proximal end to the at least one electrode. The at least one conductor is configured to electrically connect the at least one electrode to a control system. A spacer is located proximal of the at least one electrode. A movable insulation sheath is configured to move from a first position to a second position, wherein the first position covers the at least one electrode and the second position exposes the at least one electrode.

Example 17 is the device of Example 16, further comprising a platinum band located proximal of the at least one electrode.

Example 18 is the device of Example 16, further comprising a radiopaque coil, wherein a proximal face of the spacer is configured to abut a distal end of the radiopaque coil.

Example 19 is the device of Example 16, wherein the spacer is cylindrical and formed of a non-conductive material.

Example 20 is the device of Example 19, wherein the spacer is a formed of a ceramic material.

Example 21 is the device of Example 16, wherein the movable insulation sheath includes a compressible portion.

Example 22 is the device of Example 16, wherein the spacer is affixed to the movable insulation sheath.

Example 23 is the device of Example 22, further comprising a stop located on the at least one conductor, the stop being configured to abut the spacer to prevent further proximal movement of the moveable insulation sheath.

Example 24 is the device of Example 16, further comprising a radiopaque coil located in the distal portion.

Example 25 is the device of Example 24, wherein the movable insulation sheath is attached to a proximal end of the radiopaque coil, a distal end of the radiopaque coil, or both the proximal end and the distal end of the radiopaque coil.

Example 26 is the device of Example 16, wherein the distal portion includes a pre-formed curve.

Example 27 is the device of Example 26, wherein the pre-formed curve includes

a J-shape or a spiral shape.

Example 28 is the device of Example 16, wherein the distal portion includes first diameter, and the distal end includes a second diameter smaller than the first diameter.

Example 29 is the device of Example 16, wherein the movable insulation sheath includes a distal face configured to contact a target tissue.

Example 30 is a system for creating a channel in a target tissue. The system includes a perforation device having a passive electrode sheathing design. The perforation device includes an elongate body having a proximal portion including a proximal end and a distal portion including a distal end. At least one electrode is located at the distal end. The at least one electrode is configured to receive energy for piercing a target tissue. At least one conductor extends from the proximal end to the at least one electrode. A movable insulation sheath is configured to move from a first position to a second position, wherein the first position covers the at least one electrode and the second position exposes the at least one electrode. The system includes a dilator configured to dilate an opening in the target tissue created by the at least one electrode.

Example 31 is the system of Example 30, further comprising a radiofrequency generator for delivering energy to the at least one electrode.

Example 32 is a method for puncturing a target tissue. The method includes advancing a perforation device having a movable insulation sheath towards a target tissue. The method includes causing the movable insulation sheath to move from a first position covering at least one electrode to a second position exposing the at least one electrode. The method includes delivering energy to the at least one electrode and puncturing the target tissue.

Example 33 is the method of Example 32, wherein exposing the at least one electrode includes pushing a distal end of the movable insulation sheath against the target tissue.

Example 34 is the method of Example 32, wherein exposing the at least one electrode incudes moving the guidewire from a curved shape to a straight shape.

Example 35 is the method of Example 32, further comprising dilating an opening created by puncturing the target tissue.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIGS. 1A-1C are schematic illustrations of a medical procedure within a patient's heart utilizing a transseptal access system according to embodiments of the disclosure.

FIG. 2 illustrates cross-back tissue damage than occur during a procedure.

FIG. 3 illustrates through-and-through damage that can occur during a procedure including piecing an opposite side of a passageway.

FIG. 4 illustrates a perforation device having a passive electrode sheathing design, in accordance with an embodiment of the disclosure.

FIGS. 5A-5C illustrate a process of piercing body tissue using a perforation device having a passive electrode sheathing design, in accordance with an embodiment of the disclosure.

FIG. 6A illustrates a perforation device having a passive electrode sheathing design in an exposed configuration, in accordance with an embodiment of the disclosure.

FIG. 6B illustrates the perforation device of FIG. 6A having a passive electrode sheathing design in a curved sheathing configuration, in accordance with an embodiment of the disclosure.

FIG. 7A illustrates a perforation device having a passive electrode sheathing design in a covered configuration, in accordance with an embodiment of the disclosure.

FIG. 7B illustrates a perforation device having a passive electrode sheathing design in an exposed configuration, in accordance with an embodiment of the disclosure.

FIG. 8A illustrates a perforation device having a passive electrode sheathing design in a covered configuration, in accordance with an embodiment of the disclosure.

FIG. 8B illustrates a perforation device having a passive electrode sheathing design in an exposed configuration, in accordance with an embodiment of the disclosure.

FIG. 9 is a flowchart for a method of puncturing a target tissue using a perforation device with a movable insulation sheath.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

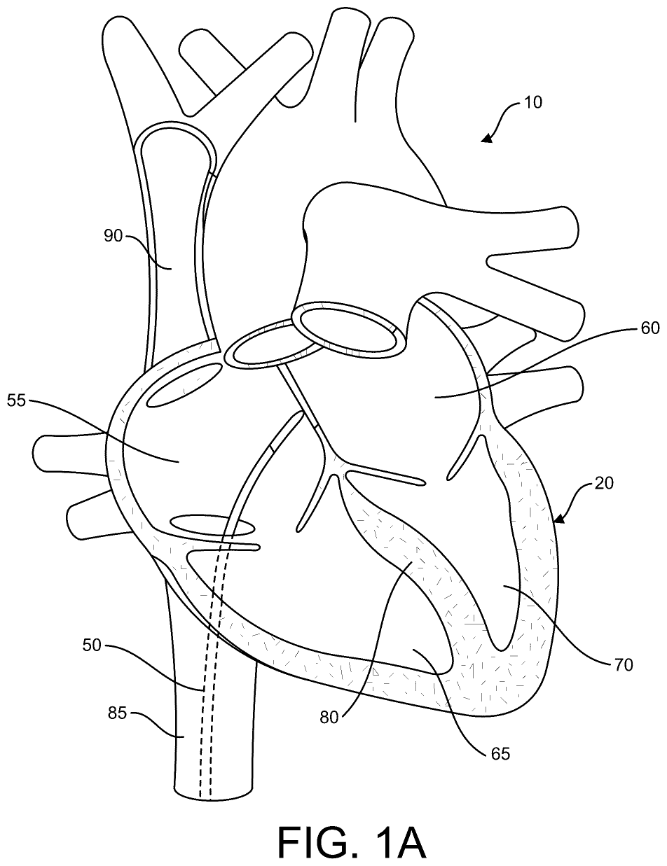

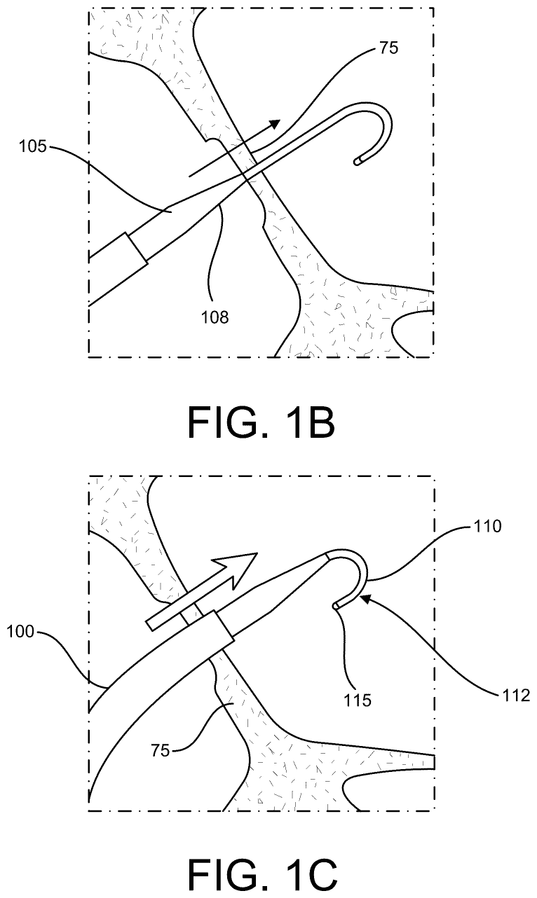

FIGS. 1A-1C are schematic illustrations of a medical procedure 10 within a patient's heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a radiofrequency (RF) perforation device 110, also known as a puncturing or perforation device, having distal end portion 112 terminating in a tip electrode 115. As shown, in the assembled use state illustrated in FIGS. 1A-1C, the RF perforation device 110 can be disposed within the dilator 105, which itself can be disposed within the sheath 100. In one embodiment in which the transseptal access system 50 is deployed into the right atrium 55 via the IVC 105, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100, and dilator 105 have been positioned in the superior vena cava, the guidewire is removed from the body, and the sheath 100 and the dilator 105 are retracted so that their distal ends are positioned in the right atrium 55. The RF perforation device 110 described can then be introduced into the dilator 105, and advanced toward the heart 20.

Subsequently, the user may position the distal end of the dilator 105 against the atrial septum 75, which can be done under imaging guidance. The RF perforation device 110 is then positioned such that electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation device 110 to the tip electrode 115 and the target site. In some embodiments, the RF generator is electrically coupled to the RF perforation device 110 using a clip that is removable coupled to a proximal portion of the perforation device 110. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 s and about 5 s.

With the tip electrode 115 of the RF perforation device 110 having crossed the atrial septum 75, the dilator 105 can be advanced forward, with the tapered distal tip portion 107 operating to gradually enlarge the perforation to permit advancement of the distal end of the sheath 100 into the left atrium 60.

In some embodiments, the distal end portion 112 of the RF perforation device 110 may be pre-formed to assume an atraumatic shape such as a J-shape (as shown in FIGS. 1B-1C), a pigtail shape or other shape selected to direct the tip electrode 115 away from the endocardial surfaces of the left atrium 60. Examples of such RF perforation devices can be found, for example, in U.S. patent application Ser. Nos. 16/445,790 and 16/346,404 assigned to Baylis Medical Company, Inc. The aforementioned pre-formed shapes can advantageously function to minimize the risk of unintended contact between the tip electrode 115 and tissue within the left atrium 60 and can also operate to anchor the distal end portion 112 within the left atrium 60 during subsequent procedural steps. For example, in embodiments, the RF perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF perforation device 110 into the left atrium 60. The anchoring function of the pre-formed distal end portion 112 inhibits unintended retraction of the distal end portion 112, and corresponding loss of access to the perforated site on the atrial septum 75, during such withdrawal.

The transseptal access system 50 may be configured to achieve a plurality of different curvatures. This is useful to allow introduction into and positioning of the system 50 at a desired location within the heart 20. For example, the various curvatures allow for achieving desired positioning of the dilator 105 and the RF perforation device 110 along a portion of the atrial septum 75.

In the various embodiments disclosed herein, the perforation device 110 does not include a hub or handle connected to a proximal portion of the wire, which could functional as a positional indicator for the perforation device, for example, by allowing a user to position such a hub or handle with respect to a corresponding structure on the outer member. Thus, to allow a user to properly position an inner member, such as a guidewire or RF perforation device 110, in relationship to the dilator 105, a system of markers may be placed on a proximal portion of the inner member to provide cues to a user. The system of markers may include a plurality of markers positioned symmetrically or asymmetrically along the proximal portion of the inner member. The markers may include a plurality of different colors or different tactile cues The plurality of different colors may include a first color and a second color that are contrasting and easily discernable from one another, for example white and blue.

The markers may provide cues to a user in order to allow for proper positioning of an inner member within an outer member during different steps of a medical procedure. For example, multiple markers can be used to indicate positioning of an inner member such that a distal tip is contained within the outer member, indicate proper positioning of a distal tip during RF delivery, indicate proper positioning of a distal for tenting tissue, indicate a recommended insertion depth into a pulmonary vein, indicate an insertion depth until max rail of the inner member, or indicate when the tip of the inner member is protruding a set distance from the outer member.

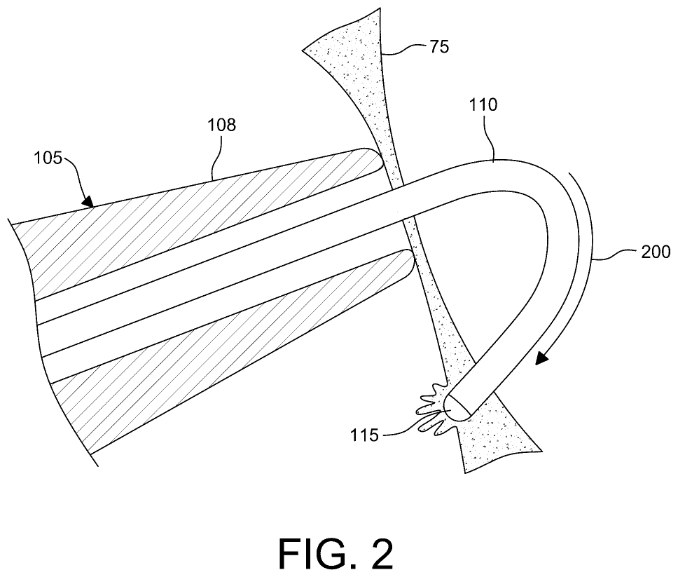

FIG. 2 illustrates cross-back tissue damage that can occur during a perforation procedure, such as a transseptal crossing, using a radiofrequency (RF) perforation device 110 having a fixed insulation and a continuously exposed tip electrode 115. FIG. 2 illustrates a target tissue, for example an atrial septum 75, that is the intended location for puncturing or piercing with the perforation device 110. In use, RF energy is applied to the tip electrode 115 and the perforation device 110 is advanced through the atrial septum 75. Upon extending into the left atrium 60, the perforation device 110 can assume an atraumatic shape such as a J-shape to direct the tip electrode 115 away from the endocardial surfaces of the left atrium 60. However, because the perforation device 110 includes a continuously exposed tip electrode 115, it is possible to damage surrounding tissue structures adjacent the target piercing location. For example, if the control system continuously applies energy to the tip electrode 115, the electrode 115 can cross-back (following arrow 200) into the atrial septum 75 and damage tissue other than the desired piercing location.

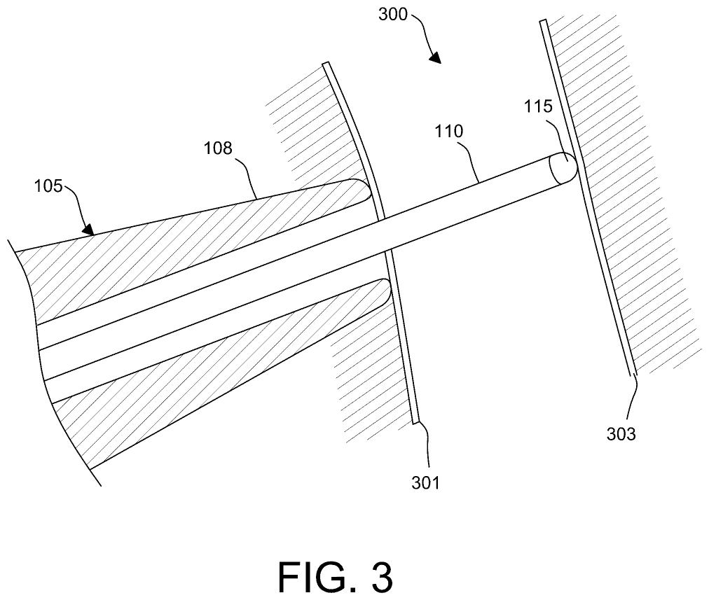

FIG. 3 illustrates piecing of an opposite side of a passageway 300 during a procedure using a radiofrequency (RF) perforation device 110 having a fixed insulation and a continuously exposed tip electrode 115. The passageway 300 can include any passageway in a body, such as an artery, vein, esophagus, heart chamber, or other hollow portion of a body. For non-curved RF perforation devices, there exists the possibility of creating a through-and-through hole from one side of the passageway 300 to the other side. This can result if too great of force is applied to the tissue prior to application of energy to the tip electrode 115. This force creates a tenting force on one side of the passageway 300, and when energy is applied to the electrode 115 the RF perforation device 110 passes through a first wall 301 into the passageway 300 and damages the opposite wall 303 of the passageway 300.

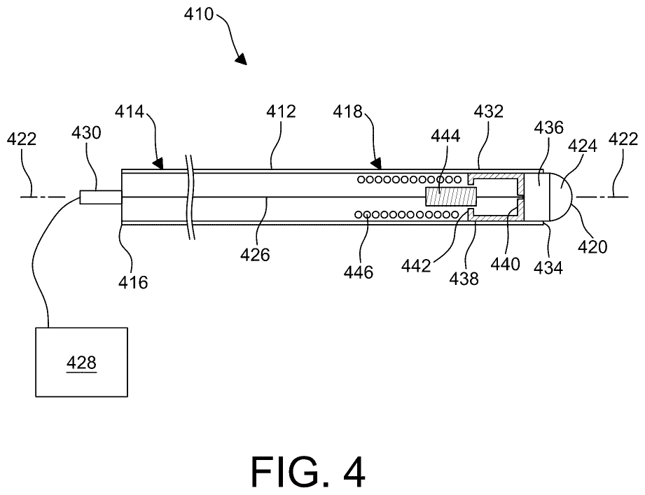

One solution for avoiding damage such as described above in FIGS. 2 and 3, includes the use of a translatable insulation that covers a distal tip RF electrode in a first configuration and then exposes the distal tip RF electrode in a second configuration. Allowing the insulation, for example a sheath formed of a non-conductive polymer, to translate along the length of a RF perforation device, safety can be increased by selectively covering and exposing an active electrode. In one aspect, a tenting interaction against target tissue results in a compressive force on the insulation that causes an unsheathing of an electrode. As the electrode is advanced through the target tissue, this compressive force dissipates, allowing the electrode to be re-sheathed and protected after it crosses the target tissue. One such embodiment is illustrated in FIG. 4.

FIG. 4 illustrates a perforation device 410, for example radiofrequency guidewire, having a passive electrode sheathing design, in accordance with an embodiment of the disclosure. The perforation device 410 includes an elongate body 412 having a proximal portion 414 including a proximal end 416 and a distal portion 418 including a distal end 420. The elongate body 412 defines a longitudinal axis 422 that generally corresponds to the geometrical centerline of the elongate body 412. At least one electrode 424 is located at the distal end 420. The at least one electrode 424 is configured to receive RF energy from a control system, for example an RF generator, for piercing a target tissue. The at least one electrode 424 is configured as a distal tip electrode and can include an atraumatic shape to avoid inadvertently piercing tissue.

At least one conductor 426 extends from the proximal end 416 to the at least one electrode 424. The at least one conductor 426 is configured to electrically connect the at least one electrode 424 to a control system 428. The proximal end 416 of the perforation device 410 may include a connector 430 to removably connect to the control system 428. The at least one conductor 426 can extend through a lumen or channel from the at least one electrode 424 to the connector 430. The at least one conductor 426 can take the form of an insulated or uninsulated wire. If uninsulated, the wire would be located in an insulated channel or lumen to prevent short circuiting.

A movable insulation sheath 432 selectively covers and uncovers the at least one electrode 424. The insulation sheath 432 includes a tubular body extending from the proximal end 416 to the distal end 420. The insulation sheath 432 can form the elongate body 412 or can be one of several layers forming the elongate body 412. The movable insulation sheath 432 is configured to move from a first position to a second position, wherein the first position covers the at least one electrode 424 and the second position exposes the at least one electrode 424. The movable insulation sheath 432 includes a distal face 434 configured to contact a target tissue. In some embodiments, the movable insulation sheath 432 is configured to translate over the entire length of the perforation device 410. For example, the entire movable insulation 432 sheath slides as a single unit. In some embodiments, the movable insulation sheath 434 is configured to translate over a portion of the perforation device 410. For example, the insulation sheath 432 may be fixed at the proximal portion 414 of the perforation device, yet free to slide at the distal portion 418.

A band 436 is located proximal of the at least one electrode 424. The band 436 connects the at least one conductor 426 the at least one electrode 424. The band 436 is electrically connected to both the at least one conductor 426 and the at least one electrode 424. The band 436 is electrically conductive. In some embodiments, the band 436 is radiopaque. The band 436 can be formed of suitable metallic materials or electroconductive polymers. In one embodiment, the band 436 is formed of platinum. The band 436 has an outer diameter that is substantially identical to the outer diameter of the at least one electrode 424.

The perforation device 410 includes a spacer 438 located proximal of the at least one electrode 424. In some embodiments, the spacer 438 is located proximal of the band 436. In some embodiments, the spacer 438 is affixed to the band 436. In other embodiments, the spacer 438 is configured to translate along the longitudinal axis 422 of the perforation device 410. The spacer 438 can have an elongated shape with a cross-section that fits within the elongate body 412. The spacer 438 has an outer diameter that is substantially identical to the outer diameter of the at least one electrode 424 and the band 436 such that there is a continuous smooth outer surface from the at least one electrode 424 to the spacer 438. In one aspect, the spacer 438 is cylindrical. In another aspect, the spacer 438 includes a polygonal cross-section, such as a square, rectangle, pentagon, hexagon, or other polygonal cross-section. The spacer 438 is formed of a non-conductive material. For example, the spacer 438 is formed of a ceramic material or a polymeric material. In one embodiment, the spacer 438 is an extruded shape. In another embodiment, the spacer 438 can be a coil formed of a wound filament, for example a round or flat wire.

The spacer 438 is affixed to the movable insulation sheath 432 such that the spacer 438 and the movable insulation sheath 432 translate together along the longitudinal axis 422 of the perforation device 410. In one embodiment the sheath 432 is affixed along the entire outer surface of the spacer 438. In another embodiment, the sheath 432 is affixed at the distal end of the spacer 438. In another embodiment, the sheath 432 is affixed at the proximal end of the spacer 438. The sheath 432 can be affixed using welding techniques, adhesives, or other means for connecting the spacer 438 to the sheath 432.

The spacer 438 includes a closed distal wall 440 and an open proximal end 442. The distal wall 440 includes an opening to allow for the passage of the at least one conductor 426. The open proximal end 442 is configured to receive a stop 444 located on the at least one conductor 426. The stop 444 is larger than the at least one conductor 426 and is configured to abut the spacer 438 to prevent further proximal movement of the moveable insulation sheath 432. In one embodiment, the stop 444 abuts the distal wall 440 by entering into the spacer 438. In another embodiment, the proximal end of the spacer 438 is closed and is configured to abut the stop 444. The stop 444 can be affixed to the at least one conductor 426 or can be an enlarged portion of the at least one conductor 426. The stop 444 can be formed of a polymeric or metallic material and can include a would filament. In some embodiments, the stop 444 can include radiopaque materials, such as tungsten.

A radiopaque coil 446 is located in the distal portion 418 of the perforation device 410. The radiopaque coil 446 provides support to the elongate tubular body 412 as well as provides a marker for viewing under imaging. The radiopaque coil 446 can be formed of any material that increases contrast under imaging and provides the requisite strength for the perforation device 410. For example, the radiopaque coil 446 can include a metal such as platinum or gold, or a polymer doped with particles such as iodine, barium, tantalum, or bismuth. The radiopaque coil 446 can be a coil formed of a wound filament, for example a round or flat wire. The spacing between turns in the radiopaque coil 446 is consistent in some embodiments, and variable in other embodiments.

In some embodiments, the movable insulation sheath 432 is free to translate along the radiopaque coil 446. In some embodiments, the movable insulation sheath 432 is attached to a proximal end of the radiopaque coil 446, a distal end of the radiopaque coil, or both the proximal end and the distal end of the radiopaque coil 446. In this configuration, the coil 446 must compress in order for the sheath 432 to move proximally to expose the at least one electrode 424. The coil 446 then expands for the sheath 432 to return to its original position covering the at least one electrode 424.

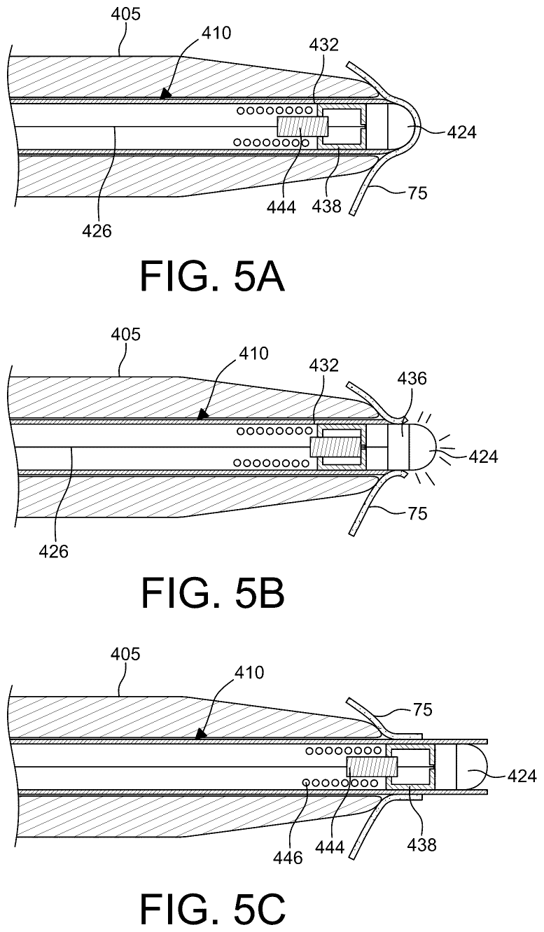

FIGS. 5A-5C illustrate a process of piercing a target body tissue using a perforation device 410 having a passive electrode sheathing design, in accordance with an embodiment of the disclosure. In FIG. 5A, the perforation device 410 is located within a dilator 405 and the distal end 420 of the perforation device 410 is pushed against the target tissue, for example an atrial septum 75. The applied force against the distal end 420 causes the target tissue 75 to tent. The tented tissue provides sufficient force to push back on distal face 434 of the moveable insulation sheath 432, causing exposure of the at least one electrode 424 located at the distal end 420 as illustrated in FIG. 5B. RF energy is delivered to the at least one electrode 424 to puncture the target tissue 75.

In some embodiments, the insulation is free to retract without being limited. In other embodiments, proximal retraction of the insulation 432 is limited by interaction of the spacer 438 with the stop 444 as illustrated in FIG. 5B. The stop 444 is positioned such that an entirety of the at least one electrode 424 is exposed when the spacer 438 contacts the stop 444. In some aspects, the stop 444 is positioned such that at least a portion of the band 436 is exposed when the spacer 438 contacts the stop 444.

Once the at least one electrode 424 passes through the opening formed in the target tissue 75, the tissue dilates over the insulation distal face 434 allowing the insulation 432 to push distally, no longer being under tension. At this point, as shown in FIG. 5C, the at least one electrode 424 is safely covered by the movable insulation sheath 432. The perforation device 410 can now be safely advanced into the heart chamber without worry of inadvertently damaging tissue adjacent the target tissue 75. Because the at least one electrode 424 is covered by the insulation sheath 432, cross-back and through-and-through damage is avoided.

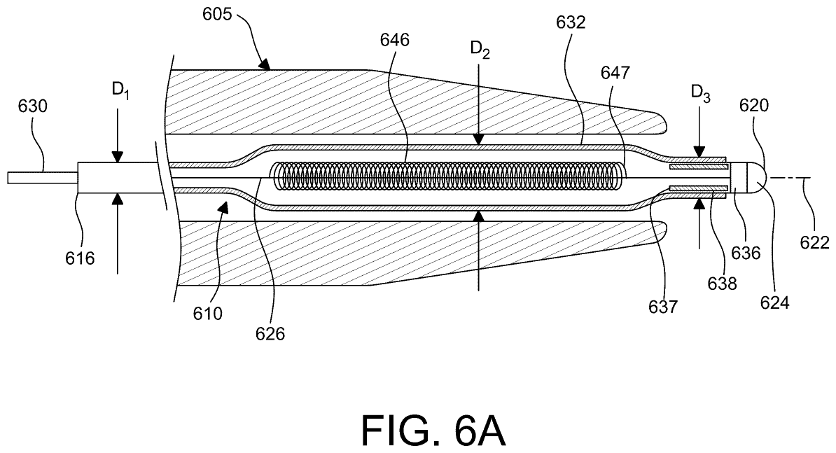

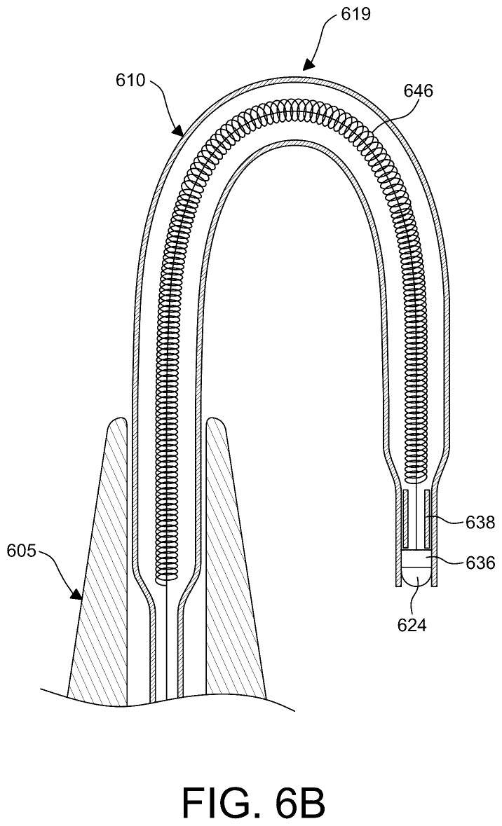

FIGS. 6A and 6B illustrate perforation device 610, such as an RF guidewire, having a pre-formed distal curve. The perforation device 610 includes an elongate body 612 having a proximal portion 614 including a proximal end 616 and a distal portion 618 including a distal end 620. In some embodiments, the elongate body 612 includes a uniform diameter from the proximal end 616 to the distal end 620. In some embodiments, the elongate body 612 includes a first diameter D1 at the proximal portion 614, a second diameter D2 at the distal portion 618, and a third diameter D3 at the distal end 620. The second diameter D2 is larger than both the first diameter D1 and the third diameter D3.

The elongate body 612 defines a longitudinal axis 622 that generally corresponds to the geometrical centerline of the elongate body 612. At least one electrode 624 is located at the distal end 620 and is configured to receive RF energy from a control system. At least one conductor 626 extends from the proximal end 616 to the at least one electrode 624. The at least one conductor 626 is configured to electrically connect the at least one electrode 624 to a control system. The proximal end 616 of the perforation device 610 may include a connector 630 to removably connect to the control system. A movable insulation sheath 632 selectively covers and uncovers the at least one electrode 624 depending on a configuration of perforation device 610.

A band 636 is located proximal of the at least one electrode 624. The band 636 connects the at least one conductor 626 the at least one electrode 624. The band 636 is electrically connected to both the at least one conductor 626 and the at least one electrode 624. The band 636 is electrically conductive. In some embodiments, the band 636 is radiopaque. The band 636 can be formed of suitable metallic materials or electroconductive polymers. In one embodiment, the band 636 is formed of platinum. The band 636 includes an outer diameter that is substantially identical to the outer diameter of the at least one electrode 624.

The perforation device 610 includes a spacer 638 located proximal of the at least one electrode 624. In some embodiments, the spacer 638 is located proximal of the band 636. The spacer 638 remains in a fixed position relative to movable insulation sheath 632 and is configured to support the distal end of the sheath 632 as the sheath 632 translates along an outer surface of the spacer 638. In some embodiments, the spacer 638 is affixed to the band 636. In other embodiments, the spacer 638 is affixed to the at least one conductor 626. In one aspect, the spacer 38 is cylindrical. The spacer 638 has an outer diameter that is substantially identical to the outer diameter of the at least one electrode 624 and the band 636 such that there is a continuous smooth outer surface from the at least one electrode 624 to the spacer 638. In another aspect, the spacer 38 includes a polygonal cross-section, such as a square, rectangle, pentagon, hexagon, or other polygonal cross-section. In one embodiment, the spacer 638 is an extruded shape. In another embodiment, the spacer 638 can be a coil formed of a wound filament, for example a round or flat wire. The spacer 638 includes a lumen which the at least one conductor 626 passes through to join the band 636.

A pre-formed curve 619, shown in FIG. 6B, is located in the distal portion 618 of the perforation device 610. In one embodiment, the pre-formed curve 619 includes a j-shape. In another embodiment, the pre-formed curve 619 includes a spiral shape. The perforation device 610 has a passive electrode sheathing design that covers the at least one electrode 624 in the pre-formed curve configuration as illustrated in FIG. 6B and exposes the at least one electrode 624 when straightened as illustrated in FIG. 6A. In FIG. 6A, the perforation device 610 is shown partially extending from a dilator 604 such that the pre-formed curve remains constrained by the dilator 604. In FIG. 6B, the distal portion 618 of the perforation device 610 is shown extending from the dilator 604 such that the pre-formed curve 619 is free to obtain the relaxed, unconstrained shape.

The perforation device 610 includes a radiopaque coil 646 that is located within the distal portion 618. The radiopaque coil 646 expands and retracts as the perforation device 610 moves from a constrained configuration to an unconstrained configuration allowing the pre-formed curve 619 to take shape. The radiopaque coil 646 is fixed to the movable insulation sheath 632 such that as the radiopaque coil 646 compresses, the movable insulation sheath 632 moves proximally exposing the at least one electrode 624. Similarly, as the radiopaque coil 646 expands, the sheath 632 moves distally covering the at least one electrode 624. In one embodiment, the radiopaque coil 646 is fixed at a distal end to the movable insulation sheath 632. In one embodiment, the radiopaque coil 646 is fixed at a proximal end to the movable insulation sheath 632. In one embodiment, the radiopaque coil 646 is fixed to the movable insulation sheath 632 at a location between the coil 646 distal end and the coil 646 proximal end.

In the unconstrained configuration, as illustrated in FIG. 6B, the spacing between the turns of the radiopaque coil 646 increases, at least along a portion thereof, thus pushing the insulation 632 forward over the at least one electrode 624. When the RF guidewire is straightened, as illustrated in FIG. 6A, the spacing between the turns decreases, thus pulling the sheath 632 proximally and exposing the at least one electrode 624.

During a perforation procedure, such as a transseptal crossing procedure, the perforation device 610 is directed towards a target tissue within a sheath or dilator 605. In this constrained configuration the at least one electrode 624 is exposed. To make a cut or puncture at the target tissue, the perforation device 610 is slightly extended from the sheath or dilator 605 such that the at least one electrode 624 contacts the target tissue. Upon application of RF energy to the at least one electrode 624, a cut or puncture is formed in the target tissue. The perforation device 610 is then extended into the cut or puncture into the heart chamber. As the perforation device 610 exits the sheath or dilator 605, the pre-formed curve 619 is unconstrained, and the perforation device 610 assumes the curved shape. This allows the radiopaque coil 646 to expand and push the movable insulation sheath 632 distally, thus covering the at least one electrode 624 and preventing inadvertent damage to tissue adjacent the target tissue. A proximal face 637 of the spacer 638 is configured to abut a distal end 647 of the radiopaque coil 646. This acts as a stop for distal translation of the movable insulation sheath 632 and can limit the amount of sheath that overhangs the at least one electrode 624.

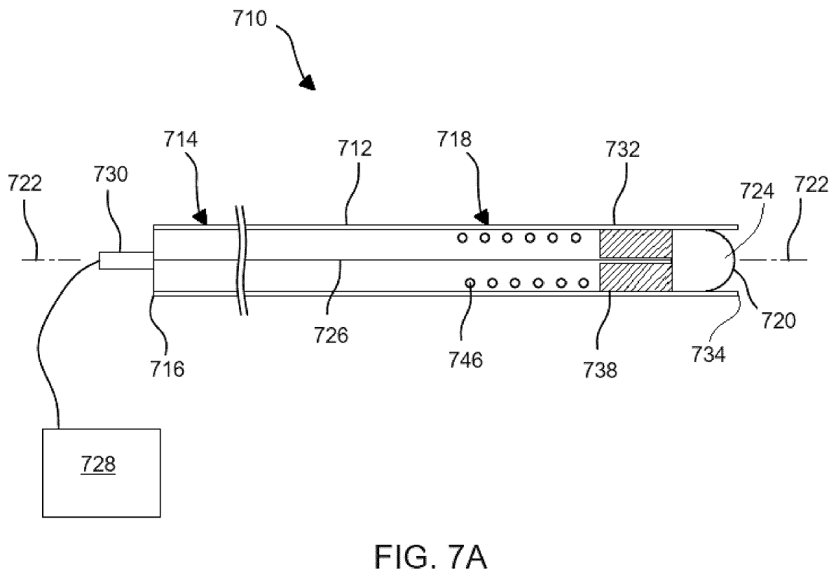

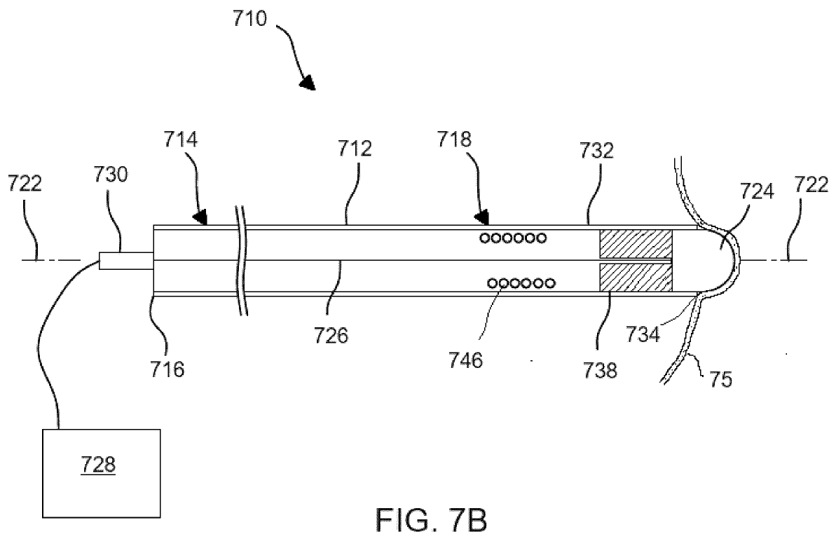

FIG. 7A illustrates a perforation device 710 having a passive electrode sheathing design in a covered configuration, in accordance with an embodiment of the disclosure. The perforation device 710 includes an elongate body 712 having a proximal portion 714 including a proximal end 716 and a distal portion 718 including a distal end 720. The elongate body 712 defines a longitudinal axis 722 that generally corresponds to the geometrical centerline of the elongate body 712. At least one electrode 724 is located at the distal end 720. The at least one electrode 724 is configured to receive RF energy from a control system 728, for example an RF generator, for piercing a target tissue. The at least one electrode 724 is configured as a distal tip electrode and can include an atraumatic shape to avoid inadvertently piercing tissue.

At least one conductor 726 extends from the proximal end 716 to the at least one electrode 724. The at least one conductor 726 is configured to electrically connect the at least one electrode 724 to a control system 728. The proximal end 716 of the perforation device 710 may include a connector 730 to removably connect to the control system 728. The at least one conductor 726 can extend through a lumen or channel from the at least one electrode 724 to the connector 730. The at least one conductor 726 can take the form of an insulated or uninsulated wire. If uninsulated, the wire would be located in an insulated channel or lumen to prevent short circuiting.

A movable insulation sheath 732 selectively covers and uncovers the at least one electrode 724. As shown in FIG. 7A the insulation sheath 732 extends over the at least one electrode 724 to protect tissue from inadvertent damage. The insulation sheath 732 includes a tubular body extending from the proximal end 716 to the distal end 720. The insulation sheath 732 can form the elongate body 712 or can be one of several layers forming the elongate body 712. The movable insulation sheath 732 is configured to move from a first position to a second position, wherein the first position covers the at least one electrode 724 and the second position exposes the at least one electrode 724. The movable insulation sheath 732 includes a distal face 734 configured to contact a target tissue. In some embodiments, the movable insulation sheath 732 is configured to translate over the entire length of the perforation device 710. For example, the entire movable insulation 732 sheath slides as a single unit. In some embodiments, the movable insulation sheath 734 is configured to translate over a portion of the perforation device 710. For example, the insulation sheath 732 may be fixed at the proximal portion 714 of the perforation device, yet free to slide at the distal portion 718.

The perforation device 710 includes a spacer 738 located proximal of the at least one electrode 724. In some embodiments, the spacer 738 is affixed to the at least one electrode. In other embodiments, the spacer 738 is affixed to the at least one conductor 726. The spacer 738 can have an elongated shape with a cross-section that fits within the elongate body 712. The spacer 738 has an outer diameter that is substantially identical to an outer diameter of the at least one electrode 724. In one aspect, the spacer 738 is cylindrical. In another aspect, the spacer 738 includes a polygonal cross-section, such as a square, rectangle, pentagon, hexagon, or other polygonal cross-section.

In some embodiments, the spacer 738 is formed of a non-conductive material. For example, the spacer 738 is formed of a ceramic material or a polymeric material. The spacer 738 includes a lumen or channel through which the at least one conductor 726 extends to the at least one electrode 724 to electrically connect the at least one electrode 724 to the control system 728. In other embodiments, the spacer 738 is a proximal extension of the at least one electrode 724 and is formed of a conductive material. In these embodiments, the at least one conductor 726 is joined to a proximal end of the spacer 738 to electrically connect the at least one electrode to the control system 728.

The spacer 738 does not move in relation to the at least one electrode 724. In other works, the spacer 738 is fixed longitudinally within the perforation device 710. The insulation sheath 732 is free to move along the spacer 738. The movable insulation sheath 732 translates over the at least one electrode 724 and the spacer 738 along the longitudinal axis 722 of the perforation device 710 in response to the distal face 734 contacting a target tissue, such as an atrial septum 75.

A radiopaque coil 746 is located in the distal portion 718 of the perforation device 710. The radiopaque coil 746 provides support to the elongate tubular body 712 as well as provides a marker for viewing under imaging. The radiopaque coil 746 can be formed of any material that increases contrast under imaging and provides the requisite strength for the perforation device 710. For example, the radiopaque coil 446 can include a metal such as platinum or gold, or a polymer doped with particles such as iodine, barium, tantalum, or bismuth. The radiopaque coil 746 can be a coil formed of a wound filament, for example a round or flat wire. The spacing between turns in the radiopaque coil 746 is consistent in some embodiments, and variable in other embodiments.

The movable insulation sheath 732 is attached to a proximal end of the radiopaque coil 746, a distal end of the radiopaque coil, or both the proximal end and the distal end of the radiopaque coil 746. In this configuration, the coil 746 is configured to compress, as shown in FIG. 7B, while the sheath 732 moves proximally to expose the at least one electrode 724. The coil 746 is configured to expand as the sheath 732 returns to its original position covering the at least one electrode 724.

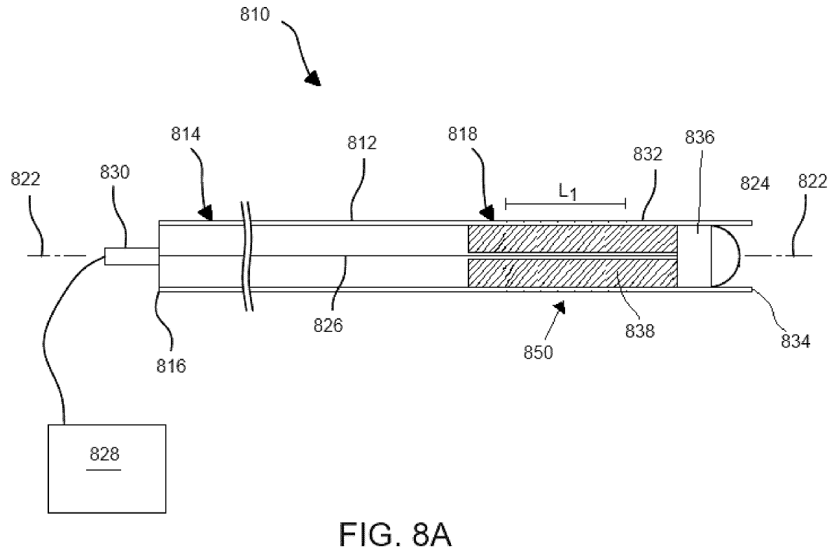

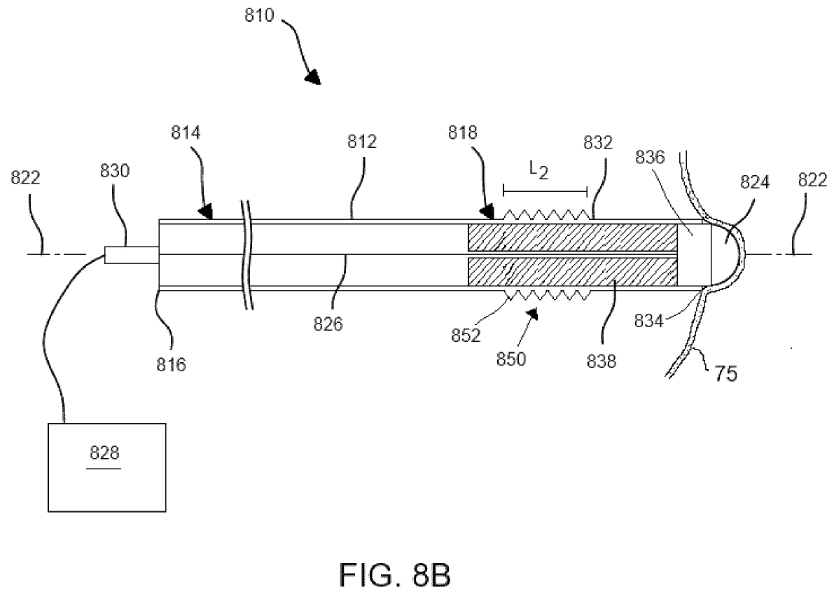

FIG. 8A illustrates a perforation device 810 having a passive electrode sheathing design in a covered configuration, in accordance with an embodiment of the disclosure. The perforation device 810 includes an elongate body 812 having a proximal portion 814 including a proximal end 816 and a distal portion 818 including a distal end 820. The elongate body 812 defines a longitudinal axis 822 that generally corresponds to the geometrical centerline of the elongate body 812. At least one electrode 824 is located at the distal end 820. The at least one electrode 824 is configured to receive RF energy from a control system, for example an RF generator, for piercing a target tissue. The at least one electrode 824 is configured as a distal tip electrode and can include an atraumatic shape to avoid inadvertently piercing tissue.

At least one conductor 826 extends from the proximal end 816 to the at least one electrode 824. The at least one conductor 826 is configured to electrically connect the at least one electrode 824 to a control system 828. The proximal end 816 of the perforation device 810 may include a connector 830 to removably connect to the control system 828. The at least one conductor 826 can extend through a lumen or channel from the at least one electrode 824 to the connector 830. The at least one conductor 826 can take the form of an insulated or uninsulated wire. If uninsulated, the wire would be located in an insulated channel or lumen to prevent short circuiting.

A movable insulation sheath 832 selectively covers and uncovers the at least one electrode 824. The insulation sheath 832 includes a tubular body extending from the proximal end 816 to the distal end 820. The insulation sheath 832 can form the elongate body 812 or can be one of several layers forming the elongate body 812. The movable insulation sheath 832 is configured to move from a first position to a second position, wherein the first position covers the at least one electrode 824 and the second position exposes the at least one electrode 824. The movable insulation sheath 832 includes a distal face 834 configured to contact a target tissue. In some embodiments, the movable insulation sheath 832 is configured to translate over the entire length of the perforation device 810. For example, the entire movable insulation 832 sheath slides as a single unit. In some embodiments, the movable insulation sheath 834 is configured to translate over a portion of the perforation device 810. For example, the insulation sheath 832 may be fixed at the proximal portion 814 of the perforation device, yet free to slide at the distal portion 818.

The insulation sheath 832 includes a compressible portion 850. The compressible portion 850 is configured to compress as a result of a force acting on the distal face 834. The compressible portion 850 is configured to have a first uncompressed length L1 shown in FIG. 8A. When the distal face 834 presses against a target tissue, such as an atrial septum 75, the compressible portion 850 compresses and has a compressed length L2 as shown in FIG. 8B. Compressed length L2 is shorter than the uncompressed length L1 such that the at least one electrode 824 is exposed when the compressible portion 850 compresses.

The compressible portion 850 is more flexible than other portions of the insulation sheath 832. In one embodiment, the compressible portion 850 is formed of a material different than the remainder of sheath 832. In other embodiments, the compressible portion 850 may be treated to reduce the flexibility compared to other portions of the sheath 832. For example, the compressible portion 850 may be treated chemically or using heat. In some embodiments, the compressible portion 850 may include a plurality of scores, cuts, or grooves to reduce the flexibility. The scores, cuts, or grooves may extend entirely through a wall of the sheath 832 or just into a portion of the wall. Additionally, the compressible portion 850 may include a bellows or other feature that allow the sheath 832 to assume a compressed configuration.

A band 836 is located proximal of the at least one electrode 824. The band 836 connects the at least one conductor 826 the at least one electrode 824. The band 836 is electrically connected to both the at least one conductor 826 and the at least one electrode 824. The band 836 is electrically conductive. In some embodiments, the band 836 is radiopaque. The band 836 can be formed of suitable metallic materials or electroconductive polymers. In one embodiment, the band 836 is formed of platinum. In some embodiments, the band 836 is a proximal portion of the at least one electrode 894 and formed integrally therewith.

The perforation device 810 includes a spacer 838 located proximal of the at least one electrode 824. In some embodiments, the spacer 838 is located proximal of the band 836. In some embodiments, the spacer 838 is affixed to the band 836. The spacer 838 can have an elongated shape with a cross-section that fits within the elongate body 812. The spacer 838 has an outer diameter that is substantially identical to an outer diameter of the band 836 and the at least one electrode 824. This provides a smooth outer surface for the moveable insulation sheath 832 to translate along. In one aspect, the spacer 838 is cylindrical. In another aspect, the spacer 838 includes a polygonal cross-section, such as a square, rectangle, pentagon, hexagon, or other polygonal cross-section. In another embodiment, the spacer 838 can be a coil formed of a wound filament, for example a round or flat wire. In some embodiments, the spacer 838 is formed of a non-conductive material. For example, the spacer 838 is formed of a ceramic material or a polymeric material. In other embodiments, the spacer 838 may be formed of a conductive material. In such configurations, the spacer 830 may be a proximal portion of the band 836 or the electrode 424.

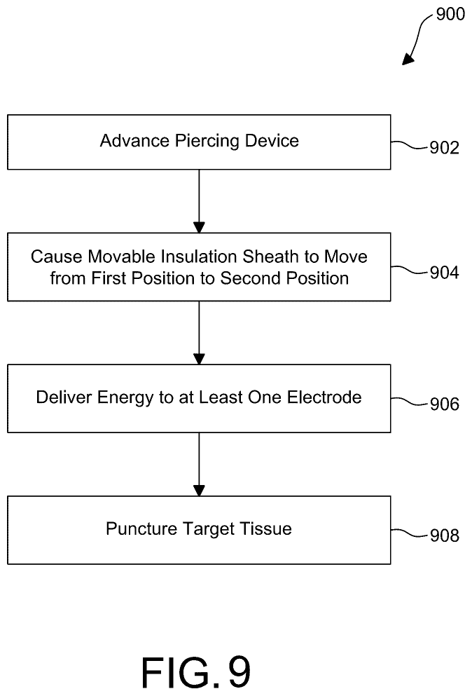

FIG. 9 is a flowchart for a method 900 of puncturing a target tissue using a perforation device with a movable insulation sheath. The method 900 includes the step 902 of advancing a perforation device having a movable insulation sheath towards a target tissue. At step 904, the movable insulation sheath is caused to move from a first position covering at least one electrode to a second position exposing the at least one electrode. In one embodiment, exposing the at least one electrode includes pushing a distal end of the movable insulation sheath against the target tissue as discussed above in FIGS. 5A-5C 7A, 7B, 8A, and 8B. In another embodiment, exposing the at least one electrode incudes moving the guidewire from a curved shape to a straight shape as discussed above in FIGS. 6A and 6B. At step 906, energy, such as RF energy, is delivered to the at least one electrode. This allows for puncturing the target tissue at step 908. After creating an opening, a dilator can be introduced in order to dilate the opening and allow access for medical devices to a body chamber.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. A perforation device having a passive electrode sheathing design, the device comprising:
an elongate body having a proximal portion including a proximal end and a distal portion including a distal end;
at least one electrode located at the distal end, the at least one electrode being configured to receive energy for piercing a target tissue;
at least one conductor extending from the proximal end to the at least one electrode, the at least one conductor configured to electrically connect the at least one electrode to a control system;
a spacer located proximal of the at least one electrode; and
a movable insulation sheath configured to move from a first position to a second position, wherein the first position covers the at least one electrode and the second position exposes the at least one electrode.

2. The device of claim 1, further comprising a platinum band located proximal of the at least one electrode.

3. The device of claim 1, further comprising a radiopaque coil, wherein a proximal face of the spacer is configured to abut a distal end of the radiopaque coil.

4. The device of claim 1, wherein the spacer is cylindrical and formed of a non-conductive material.

5. The device of claim 4, wherein the spacer is a formed of a ceramic material.

6. The device of claim 1, wherein the movable insulation sheath includes a compressible portion.

7. The device of claim 1, wherein the spacer is affixed to the movable insulation sheath.

8. The device of claim 7, further comprising a stop located on the at least one conductor, the stop being configured to abut the spacer to prevent further proximal movement of the moveable insulation sheath.

9. The device of claim 1, further comprising a radiopaque coil located in the distal portion.

10. The device of claim 9, wherein the movable insulation sheath is attached to a proximal end of the radiopaque coil, a distal end of the radiopaque coil, or both the proximal end and the distal end of the radiopaque coil.

11. The device of claim 1, wherein the distal portion includes a pre-formed curve.

12. The device of claim 11, wherein the pre-formed curve includes a J-shape or a spiral shape.

13. The device of claim 1, wherein the distal portion includes first diameter, and the distal end includes a second diameter smaller than the first diameter.

14. The device of claim 1, wherein the movable insulation sheath includes a distal face configured to contact a target tissue.

15. A system for creating a channel in a target tissue, the system comprising:
a perforation device having a passive electrode sheathing design, the perforation device comprising:
an elongate body having a proximal portion including a proximal end and a distal portion including a distal end;
at least one electrode located at the distal end, the at least one electrode being configured to receive energy for piercing a target tissue;
at least one conductor extending from the proximal end to the at least one electrode; and
a movable insulation sheath configured to move from a first position to a second position, wherein the first position covers the at least one electrode and the second position exposes the at least one electrode; and
a dilator configured to dilate an opening in the target tissue created by the at least one electrode.

16. The system of claim 15, further comprising a radiofrequency generator for delivering energy to the at least one electrode.

17. A method for puncturing a target tissue, the method comprising:
advancing a perforation device having a movable insulation sheath towards a target tissue;
causing the movable insulation sheath to move from a first position covering at least one electrode to a second position exposing the at least one electrode;
delivering energy to the at least one electrode; and
puncturing the target tissue.

18. The method of claim 17, wherein exposing the at least one electrode includes pushing a distal end of the movable insulation sheath against the target tissue.

19. The method of claim 17, wherein exposing the at least one electrode incudes moving the guidewire from a curved shape to a straight shape.

20. The method of claim 17, further comprising dilating an opening created by puncturing the target tissue.
